(19) United States
(12) Patent Application Publication
(10) Pub. No.: US 2025/0360162 A1
(43) Pub. Date: Nov. 27, 2025

(54) Naphthoic Acid-Derived Polymers as Antimicrobial Agents

(71) Applicant: UNIVERSITY OF SOUTH CAROLINA, COLUMBIA, SC (US)

(72) Inventors: CHUANBING TANG, COLUMBIA, SC (US); LEMAN KURNAZ, COLUMBIA, SC (US)

(21) Appl. No.: 19/086,671

(22) Filed: Mar. 21, 2025

Related U.S. Application Data
(60) Provisional application No. 63/651,156, filed on May 23, 2024.

(51) Int. Cl. A61K 31/78; A61L 15/44; A61P 31/04

(52) U.S. Cl. CPC A61K 31/78; A61P 31/04; A61L 15/44

(57) ABSTRACT

The present disclosure is generally directed to antimicrobial polymers, and methods of preparation thereof. In embodiments, an antimicrobial polymer may comprise a polymeric moiety, an aromatic moiety comprising a naphthoic acid derivative bonded to the polymeric moiety, a pendant group comprising a cation and a linking group bonded to the pendant group and aromatic moiety. Methods of preparation include reacting naphthoic acid or a derivative thereof with a polymer to form a primary intermediate, reacting an alcohol group on the primary intermediate with an acyl halide to form a secondary intermediate, reacting a terminal halide on the acyl halide to form a cationic group on the secondary intermediate.

### CROSS REFERENCE TO RELATED APPLICATION

This application claims filing benefit of U.S. Provisional Patent Application Ser. No. 63/651,156 having a filing date of May 23, 2024, which is incorporated herein by reference for all purposes.

### FEDERAL RESEARCH STATEMENT

This invention was made with Government support under Contract No. R01AI149810, awarded by the National Institutes of Health. The Government has certain rights in the invention.

### BACKGROUND

Bacterial infection from multi-drug resistant (MDR) bacteria has caused 70,000 deaths per year, and it was estimated that without intervention, this number could reach 10 million globally by 2050. Antimicrobial resistance (AMR), the ability of a pathogen to survive after being exposed to an antimicrobial agent, threatens the global health with increasing outcomes of morbidity and mortality. Infections by gram-negative bacteria are the most problematic for current antibiotics, largely because they consist of three principal layers of defense, inner membrane, peptidoglycan cell wall, and outer membrane. Extensively drug-resistant (XRD) and pan drug-resistant (PDR) terminologies were recently introduced to describe the highly resistant gram-negative bacteria such as Klebsiella pneumoniae, Pseudomonas aeruginosa, and Acinetobacter baumannii. Those major representative strains can be resistant to almost all except the last resort antibiotics, which also have limited treatment options because of possible toxicity. Therefore it is urgent to develop novel strategies or alternatives to conventional antibiotics for tackling the AMR problem.

### SUMMARY

The present disclosure is generally directed to antimicrobial polymers and methods of formation thereof. In an embodiment of the present disclosure an antimicrobial polymer is described, the antimicrobial polymer comprising a polymeric moiety, an aromatic moiety comprising a naphthoic acid derivative bonded to the polymeric moiety, a pendant group comprising a cation and a linking group bonded to the pendant group and aromatic moiety.

In an embodiment of the present disclosure, a method for forming an antimicrobial polymer is disclosed, the method comprising reacting naphthoic acid or a derivative thereof with a polymer to form a primary intermediate, reacting an alcohol group on the primary intermediate with an acyl halide to form a secondary intermediate, reacting a terminal halide on the acyl halide to form a cationic group on the secondary intermediate.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 is a flow diagram showing a few exemplary antimicrobial polymers and the synthetic routes therefore;

FIG. 2 is ¹H NMR of a polymeric moiety contemplated by the present disclosure;

FIG. 3A-3E are a series of ¹H NMR spectrums of the polymeric moiety with a side chain thereof bonded to a 2-naphthoic acid through an ester linkage (FIG. 3A) to form an NA polymer, the NA polymer bonded to a four carbon chain length linking group (3B), a cationic group at the terminal position on the linking group of the NA polymer (3C), the NA polymer of 3A bonded to a six carbon chain length linking group (3D), a cationic group at the terminal position on the linking group of the NA polymer of 3D (3E);

FIGS. 3F and 3G are FT-IR spectrums showing the disappearance of an —OH stretch before and after esterification of alcohol groups on the polymer sidechain;

FIG. 4A-4E are a series of ¹H NMR spectrums of the polymeric moiety with a side chain thereof bonded to a 2-naphthoic acid through an ester linkage (FIG. 4A) to form an HNA polymer, the HNA polymer bonded to a four carbon chain length linking group (4B), a cationic group at the terminal position on the linking group of the HNA polymer (4C), the HNA polymer of FIG. 4A bonded to a six carbon chain length linking group (4D), a cationic group at the terminal position on the linking group of the HNA polymer of FIG. 4D (4E);

FIGS. 4F and 4G are FT-IR spectrums showing the disappearance of an —OH stretch before and after esterification of alcohol groups on the polymer sidechain;

FIG. 5A-5E are a series of ¹H NMR spectrums of the polymeric moiety with a side chain thereof bonded to a 2-naphthoic acid through an ester linkage (FIG. 5A) to form an DHNA polymer, the DHNA polymer bonded to a four carbon chain length linking group (5B), a cationic group at the terminal position on the linking group of the DHNA polymer (5C), the DHNA polymer of 5A bonded to a six carbon chain length linking group (5D), a cationic group at the terminal position on the linking group of the DHNA polymer of 5D (5E);

FIGS. 5F and 5G are FT-IR spectrums showing the disappearance of an —OH stretch before and after esterification of alcohol groups on the polymer sidechain;

FIG. 6 is a table showing antimicrobial polymers and names thereof contemplated by the present disclosure;

FIG. 7 is a table showing example molecular weights of the antimicrobial polymers shown in FIG. 6;

FIG. 8 is a chromatogram showing the elution time for the polymer moiety vs. that of an antimicrobial polymer of the present disclosure. The peak elution of the antimicrobial polymer occurred at 25 minutes, whereas the peak elution of the antimicrobial polymer occurred at 33 minutes;

FIG. 9 is a table showing the minimum inhibitory concentration of the antimicrobial polymer for various microbes;

FIG. 10 is a table showing the minimum inhibitory concentration and selectivity index of the antimicrobial polymer for various microbes;

FIGS. 11A-11D is a series of four bar charts showing the response of various species against varying concentrations of the antimicrobial polymer according to the minimum inhibitory concentration;

FIGS. 12A-12C is a collection of data regarding the relative hemolysis, relative cytotoxicity and selectivity index of antimicrobial polymers of the present disclosure;

FIGS. 13A-13D is a series of graphs showing the colony forming units of various microbes when subjected to varying concentrations of antimicrobial polymers as a function of time;

FIG. 14 depicts a bar chart showing the inhibition of microbe growth after administration of an antimicrobial polymer of the present disclosure;

FIGS. 15A and 15B depicts bar charts biofilm biomass and colony forming units as a function of a control, multiples of MIC for an antimicrobial polymer and polymyxin-B;

FIG. 16 is a microgram showing the eradication of a biofilm for two concentrations of 3-QAC-Hex and one concentration of polymyxin-B, as well as a control sample;

FIGS. 17A-17F is a series of graphs showing membrane permeabilization/disruption experiments against MDR-K. pneumoniae and MDR-P. aeruginosa. Membrane depolarization plots after exposure to 1-QAC-Hex (FIG. 17A), outer membrane permeabilization (FIG. 17C) and inner membrane permeabilization (FIG. 17E) are shown for MDR-K. pneumoniae. Membrane depolarization (FIG. 17B), outer membrane permeabilization (FIG. 17D) and inner membrane permeabilization (FIG. 17F) in MDR-P. aeruginosa after exposure to 3-QAC-Hex;

FIG. 18 is a series of micrograms showing the destruction of microbes before and after treatment with 3-QAC-Hex;

FIGS. 19A and 19B are a series of bar charts showing the relatively smaller development of resistance to the antimicrobial polymers by various microbe species as compared to traditional antibiotics.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

As used herein, the term “or” is inclusive unless stated otherwise. For instance, if a computer requires A or B to be true in order to perform operation C, the case of both A and B being true will satisfy the condition necessary for C to occur. That is, “or” is inclusive of A, B, and A and B.

In general, the present disclosure is directed to antimicrobial polymers, their methods of formation and uses. In embodiments of the present disclosure, the antimicrobial polymer comprises a polymer moiety comprising a side chain, with said side chain comprising aromatic moiety comprising a naphthoic acid derivative. Further, the side chain may comprise a cationic group.

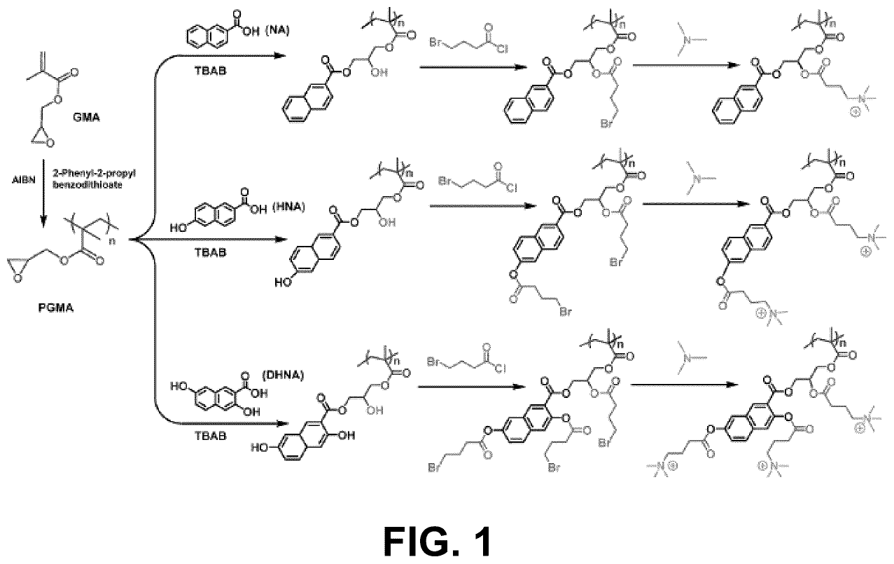

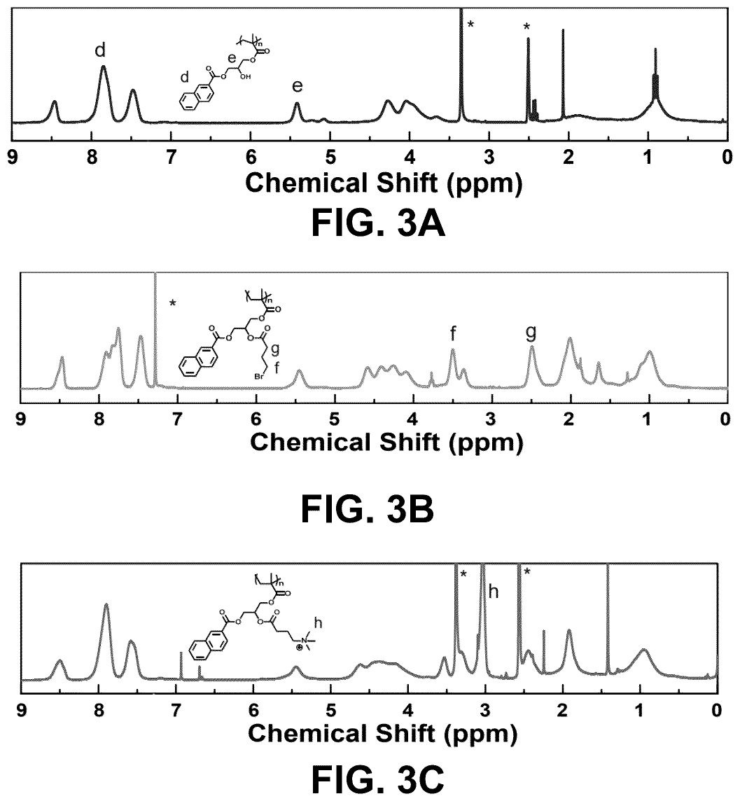

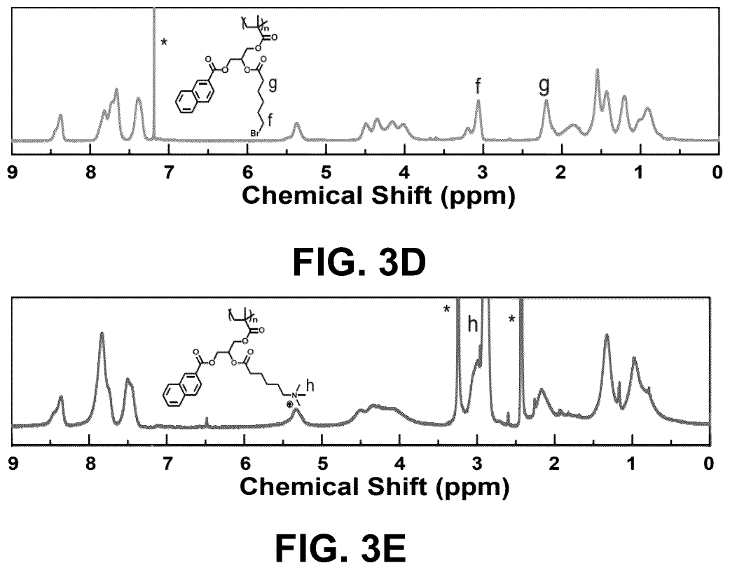

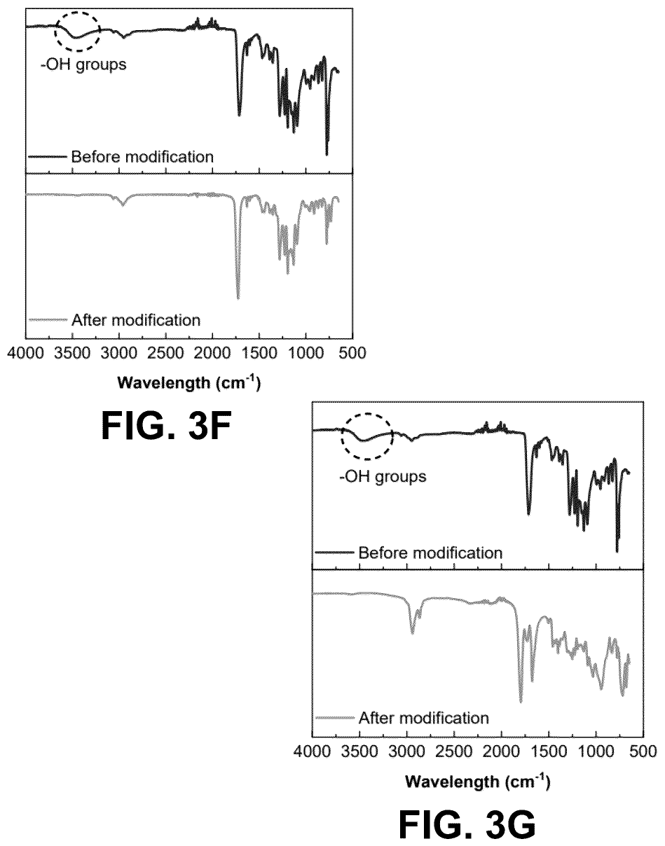

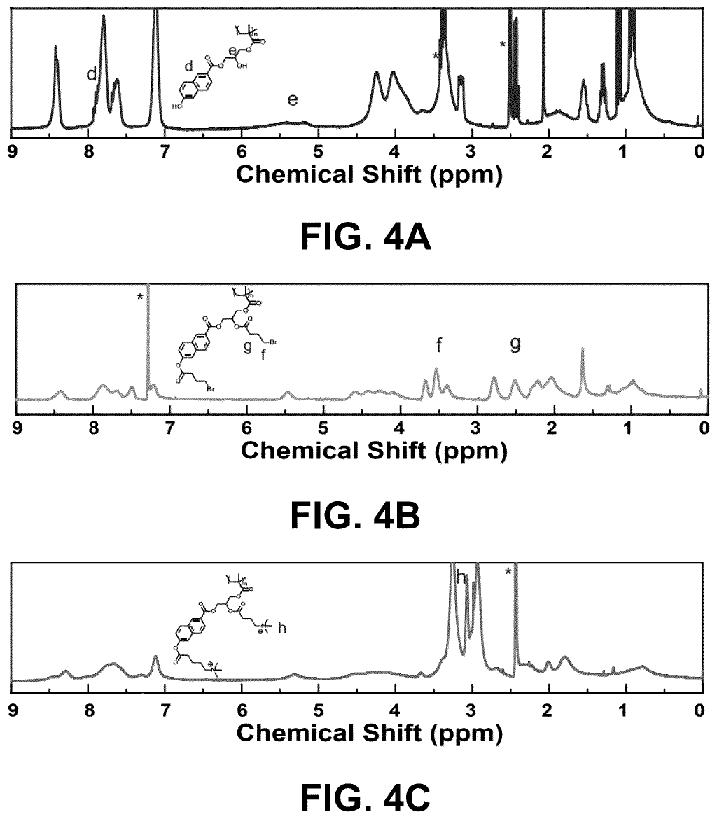

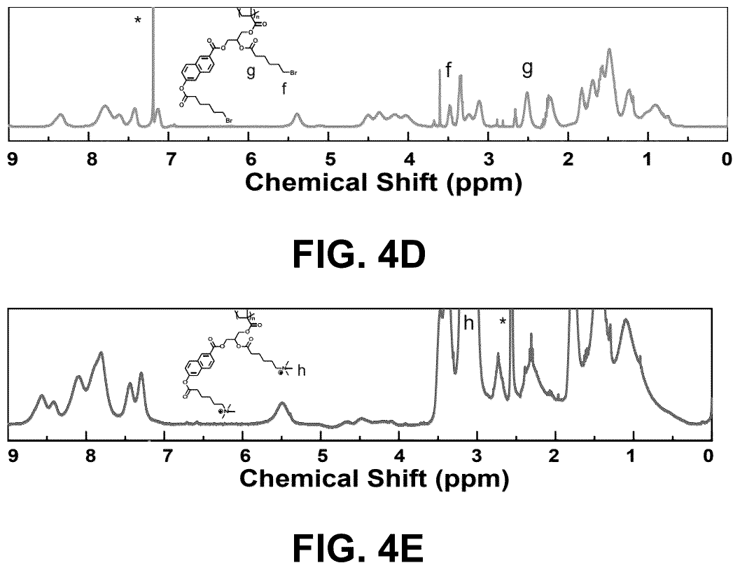

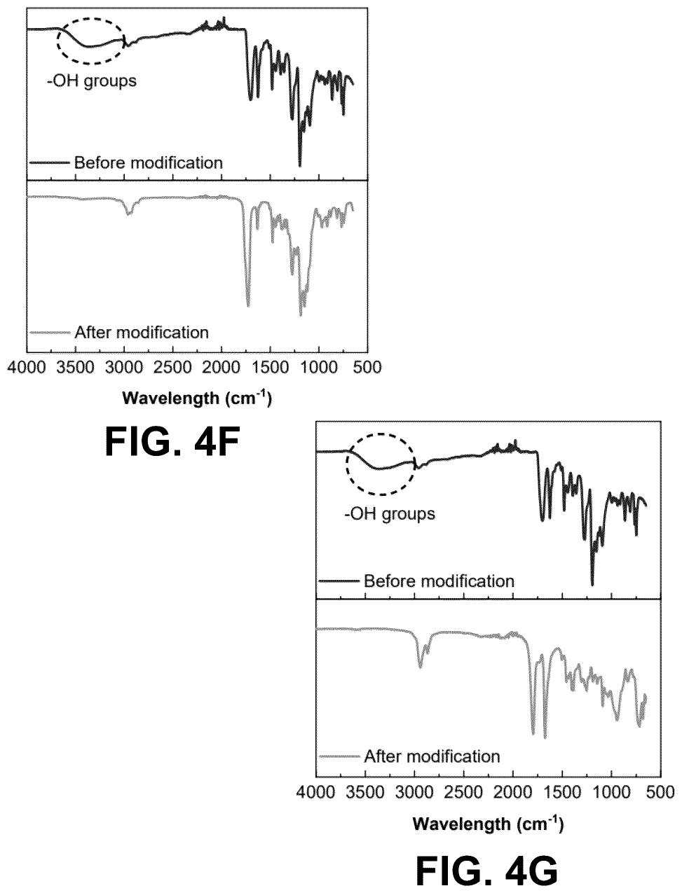

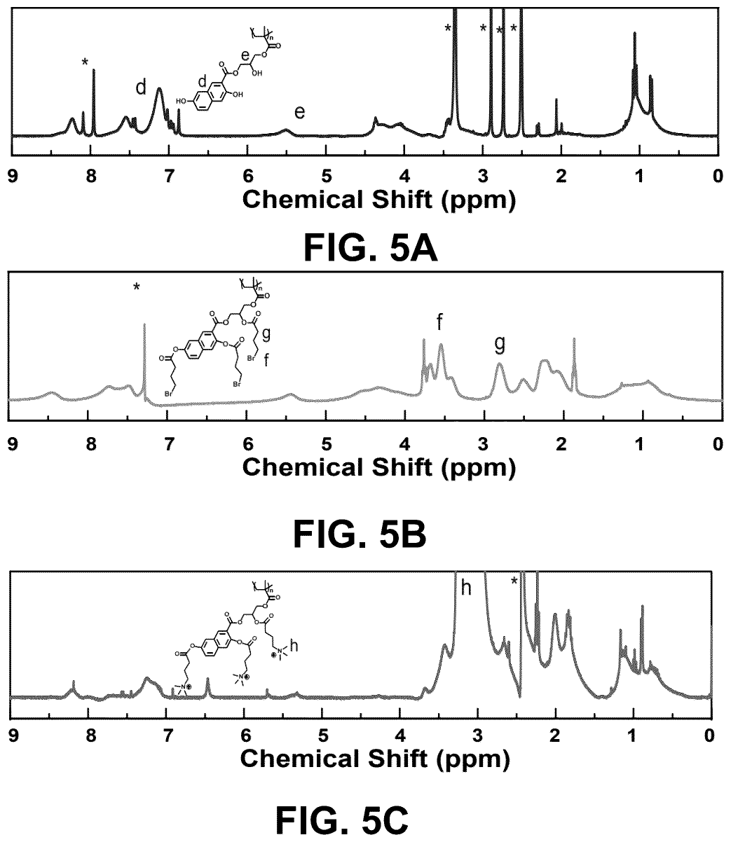

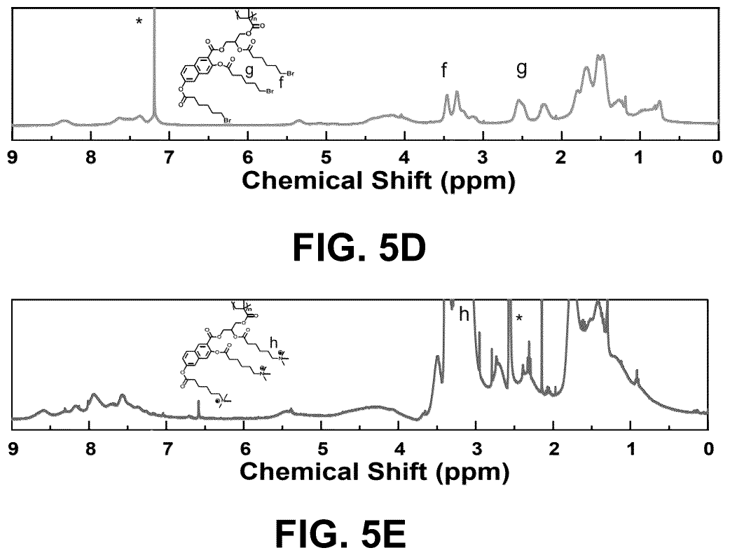

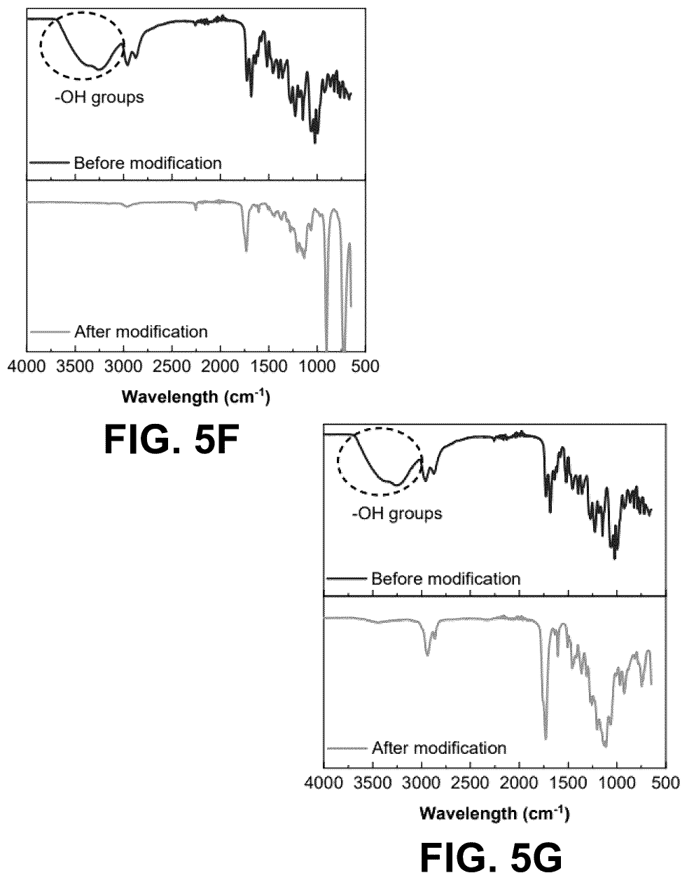

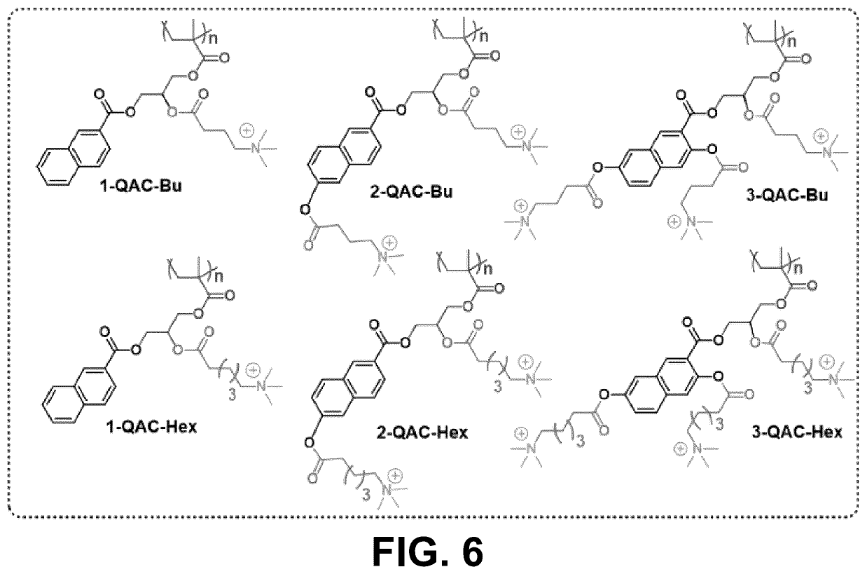

The presently described antimicrobial polymers may be formed by the methods described in the following paragraphs. In general, the formation of antimicrobial polymers of the present disclosure includes the steps of 1) reacting an aromatic moiety comprising a naphthoic acid or derivative thereof with a polymer side chain of a polymer moiety to form a primary intermediate, 2) reacting an alcohol group on the primary intermediate with an linking group comprising an acyl halide to form a secondary intermediate, 3) reacting a terminal halide to form an pendant on the linking group to form a pendant group comprising an organic cation. FIG. 1 and FIG. 6 show a flowchart of the exemplary method, and antimicrobial polymers which can be synthesized therefrom. FIGS. 3A-3G, 4A-4G and 5A-5G provide NMR (nuclear magnetic resonance) and FTIR (Fourier transform infra-red spectroscopy) spectra of intermediate steps of the synthesis of antimicrobial polymers. In general, the antimicrobial polymers described in FIGS. 3A-3G are NA polymers. That is, the aromatic moiety comprises naphthoic acid. FIGS. 3B, 3C and 3F pertain to the four carbon chain length linking group, whereas FIGS. 3D, 3E and 3G pertain to the six carbon chain length linking group. The same figure scheme is applicable to FIGS. 4A-4G and 5A-5G, with FIGS. 4A-4G pertaining to HNA polymers, and FIGS. 5A-5G pertaining to DHNA polymers.

While various methods for the addition of an acidic moiety (e.g., a naphthoic acid) to a polymer backbone may be known, in one embodiment, the acidic moiety may be added by acid-catalyzed epoxide ring-opening. The reaction of an acidic moiety and an epoxide may be seen in Scheme I below.

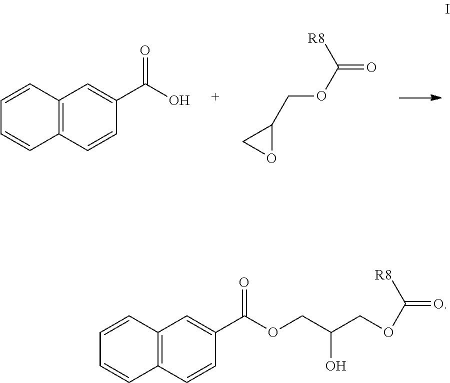

While the above scheme makes use of glycidal ester, the reaction of Scheme I does not require the use of a glycidal ester, merely an epoxide functional group. Furthermore, R8 may be a polymer as will be discussed below. The reaction may take place in a suitable solvent including, ketones, sulfoxides, ethers or mixtures thereof. In general, when using acid-catalyzed epoxide ring-opening, the solvent may be non-protic. Furthermore, the reaction between the naphthoic acid or derivative thereof and the polymer side chain may utilize a catalyst, such as a phase transfer catalyst. In embodiments, the phase transfer catalyst may comprise tetrabutylammonium bromide (TBAB).

The naphthoic acid or derivative thereof may be present in the reaction mixture at a molar % of between 30 and 80 molar %, such as between 45 and 65 molar % as a function of the total moles represented by the polymer backbone and phase transfer catalyst. In embodiments, the phase transfer catalyst may be present in a molar % of between 0 and 5 molar %, such as between 0.1 and 3 molar %.

The reaction mixture may be heated to temperatures greater than room temperature, such as between 25° C. to 150° C., such as between 40° C. and 100° C., such as between 60° C. and 90° C. Further, the reaction may be carried out over a period of time between 6 hours and 5 days, such as between 12 hours and 3 days, such as between 1 day and 2 days. During the reaction, the atmosphere may be an inert atmosphere, such as a nitrogen atmosphere, or a noble gas atmosphere, such as an argon atmosphere. After the reaction, the primary intermediate of the antimicrobial polymer may be formed.

The present disclosure is not so limited to be restricted to epoxide-based coupling mechanisms. For example, in place of an epoxide moiety, the polymer side chain may comprise an alcohol, with which the naphthoic acid or derivative thereof may form an ester.

After the reaction between the naphthoic acid or derivative thereof and the polymer backbone or polymer moiety, the primary intermediate may be cleaned of any side-products or unreacted reagents. Generally, cleaning of the primary intermediate may be carried out by cooling the reaction mixture, and precipitating the primary intermediate with a solvent wash. The step of solvent washing may comprise a plurality of solvent washing steps, such as greater than 1 washing step, such as greater than 2 washing steps, such as greater than 3 washing steps. The solvent may comprise an alcohol, ketone, ether or mixtures thereof or any suitable solvent in which the primary intermediate is insoluble.

The secondary intermediate may be formed by reaction of the primary intermediate with an acyl halide having a terminal halide. The reaction scheme for the formation of the secondary intermediate is shown below in Scheme II.

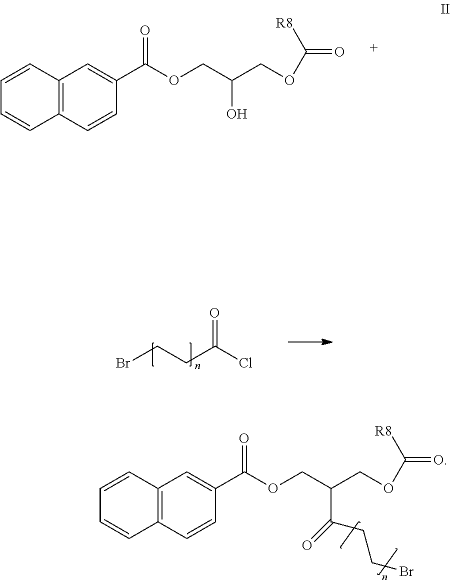

As shown above in Scheme II, the reaction of the acyl halide and the primary intermediate causes the formation of the secondary intermediate, as well as a produced acid, in this case being hydrochloric acid. In the above Scheme II, n may represent a number between 1 and 4. In general, the acyl halide can be present in the reaction flask with the primary intermediate in an excess. For instance, the reaction between the acyl halide and the primary intermediate may comprise adding more than one equivalent of the acyl halide, such as more than 3 equivalents of the acyl halide, such as more than 8 equivalents of the acyl halide.

The reaction between the primary intermediate the acyl halide may be performed by first dissolving the primary intermediate in a cooled solvent, such as dimethyl formamide (DMF), in a reaction vessel. Such solvents are not particularly limited, but may comprise aldehydes, ketones, ethers, alcohols, or mixtures thereof. The solvent into which the primary intermediate is added may initially be kept under ice. The acyl halide may then be added into the reaction vessel with vigorous stirring. The temperature may slowly be raised to a temperature between 20° C. and 80° C., such as between 30° C. and 60° C., in order for the reaction to proceed to completion, thereby forming the secondary intermediate. As with the primary intermediate above, the secondary intermediate may be cleaned by repeated washings with a solvent in which the secondary intermediate is insoluble.

The final antimicrobial polymer may be formed by the substitution of the terminal halide with a nucleophile, the terminal halide acting as a leaving group. In the below Scheme III, the nucleophile is trimethyl amine. However, as will be discussed below, it will be apparent to one of skill in the art that the nucleophile may be any such chemical so as to form an organic cation by nucleophilic substitution.

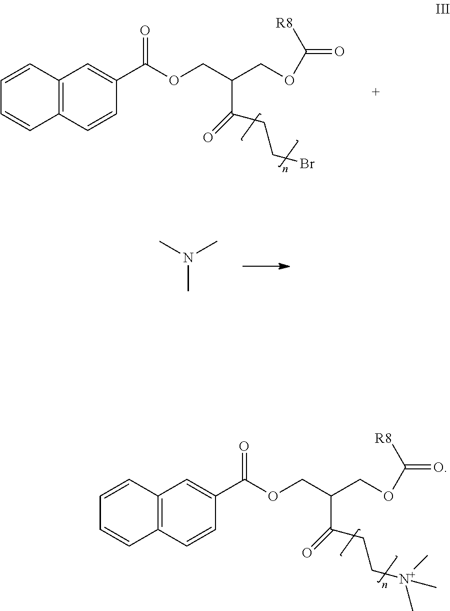

The reaction between the primary intermediate and the acyl halide may be carried out under heating, such as temperatures between 20° C. and 80° C., such as between 30° C. and 70° C., such as between 40° C. and 60° C. Further, the reaction may be left at elevated temperatures for a period of time between 6 hours and 2 days, such as between 12 hours and 1 day. Similarly to as above, the antimicrobial polymer may be dissolved in methanol and precipitated with a series of washings with a solvent in which the antimicrobial polymer is insoluble, such as tetrahydrofuran (THF).

The nucleophile may be added to the reaction with the secondary intermediate in an excess, as with the acyl halide above. For instance, the nucleophile may be added in an amount greater than 3 equivalents of the secondary intermediate, such as greater than 5 equivalents of the secondary intermediate.

While the above Scheme III shows only one organic cationic group, the present disclosure contemplates the formation of antimicrobial polymers with a plurality of organic cationic groups. For instance, as discussed below, various alternative reagents may be supplied for use in the above reaction schemes.

The naphthoic acid or derivative thereof may have the general structure shown below in Structure I.

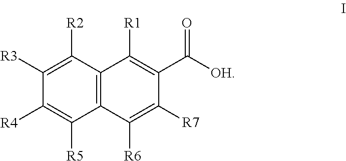

In the above structure representing the aromatic moiety comprising naphthoic acid or derivative thereof, the groups represented by R1-R7 may independently comprise hydrogen, an alkane such as a methyl, ethyl or propyl group, an ester, or an alcohol. Without wishing to be limited, specifically contemplated derivates of naphthoic acid in the present disclosure include 6-hydroxy-2-napthoic acid (HNA) and 3,7-dihydroxy-2-naphthoic acid. In general, however, it will be understood by one of skill in the art that addition of an alcohol group at any one of the R1-R7 positions may allow for the primary intermediate to react with two or more acyl halides. This increased substitution may allow for the secondary intermediate to react with a plurality of nucleophiles in order to form an antimicrobial polymer with a plural valency.

Similarly, shown above in Scheme 2, the length of the linking group or acyl halide chain may be varied. In embodiments, the acyl halide may have a primary chain length of between 2 and 10 carbons, such as between 3 and 7 carbons, such as between 4 and 6 carbons. In embodiments, the acyl halide may have a primary chain length of between 3 and 6 carbons. Additionally, the acyl halide portion may comprise any good halide leaving group, such as chlorine, bromine or iodine. As with the composition of the acyl halide, the terminal halide may comprise chlorine, bromine or iodine.

The nucleophile may comprise a tertiary amine, such as in trimethyl amine. In embodiments, the nucleophile may comprise triethyl amine, or a tertiary amine with varying substituents. Alternatively, the nucleophile may comprise a cation of sulfur or phosphorus, such as in sulfonium or phosphonium. Furthermore, the antimicrobial polymer described above may comprise a mixture of quaternary amines, sulfoniums and phosphoniums. The cationic group may be present as a pendant group bonded to the linking group. Further, the cationic group may be present in singular form on a antimicrobial polymer sidechain, or may be present in a plurality of on an antimicrobial polymer sidechain. For instance, one of skill in the art can envisage utilizing a naphthoic acid derivative with a large number of alcohol substitutions to increase the number of cationic groups present on the end antimicrobial polymer. Such an antimicrobial polymer may comprise side chains comprising between 1 and 10 cationic groups, such as between 2 and 6 cationic groups, such as between 3 and 4 cationic groups.

Finally, the polymer backbone or polymer moiety, represented by R8 in Schemes I-III, of the antimicrobial polymer may comprise polyalkanes, such as poly(methyl methacrylate), polyesters, polyamides, polypeptides or combinations thereof. The polymer backbone may be synthesized by a variety of methods including, but not limited to, reversible addition fragmentation chain transfer. In embodiments, controlling the means by which the polymer is synthesized can allow a practitioner of the art to control various characteristics of the resultant polymer. Such characteristics may include, but are not limited to, molecular weight, poly dispersity or polymer composition. In general, the polymer backbone of the antimicrobial polymer may be selected so as to have a molecular weight between 500 g/mol and 500,000 g/mol, such as between 1,000 g/mol and 100,000 g/mol, such as between 2,000 g/mol and 50,000 g/mol, such as between 5,000 g/mol and 40,000 g/mol.

The polydispersity of a plurality of the antimicrobial polymers may be greater than 0.1, such as greater than 0.3, such as greater than 0.8, such as greater than 2, such as greater than 10. The polydispersity of the plurality of antimicrobial polymers may be less than 9, such as less than 1.5, such as less than 0.7, such as less than 0.4.

In embodiments, the antimicrobial polymer may have a selectivity index of between 15 and 1000, such as between 30 and 550. In embodiments, the antimicrobial polymer may have an HC50 of greater than 800 μg/mL, such as greater than 1000 μg/mL. The HC50 is a concentration wherein 50% of the cells in a sample have undergone hemolysis.

The composition of the polymer is not particularly limited. As stated above, the antimicrobial polymer may comprise a polymer backbone comprising poly(methyl methacrylate), polyesters, polyamides or polypeptides. Further, the polymer backbone may comprise a copolymer, such as a copolymer of poly(methyl methacrylate) and another polymer, such as polyester. Furthermore, the degree of functionalization of the side chains is not particularly limited. The degree to which the side chains of the polymer backbone is functionalized may be a function of the amount of reagent used in each step of the synthesis scheme.

Without wishing to be limited to any particular chemical structure, the present disclosure contemplates the below structures for use as an antimicrobial polymer.

A first antimicrobial polymer, hereby referred to as “1-QAC-Bu” has the structure shown below:

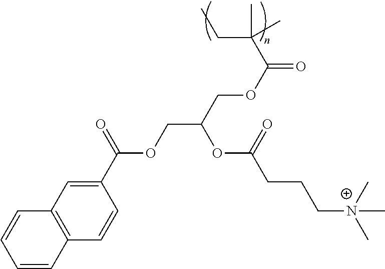

A second antimicrobial polymer, hereby referred to as “1-QAC-Hex”, has the structure shown below:

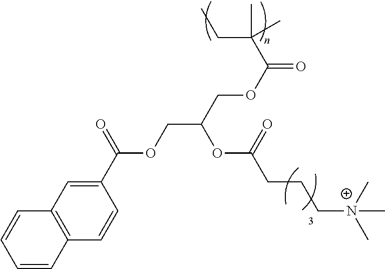

A third antimicrobial polymer, hereby referred to as “2-QAC-Bu”, has the structure shown below:

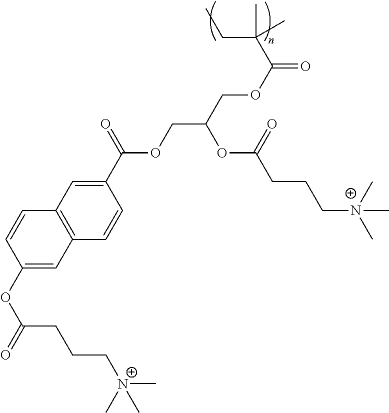

A fourth antimicrobial polymer, hereby referred to as 2-QAC-Hex”, has the structure shown below:

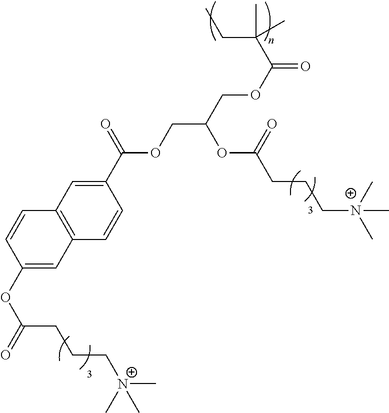

A fifth antimicrobial polymer, referred to as “3-QAC-Bu”, has the structure shown below:

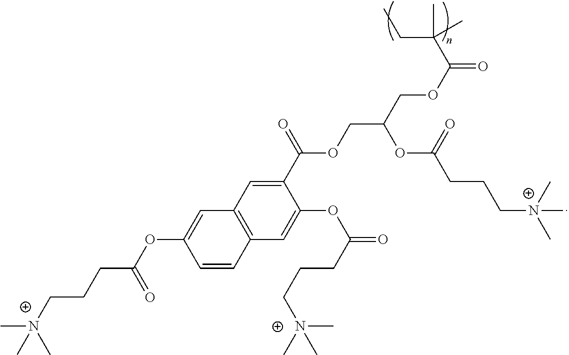

A sixth antimicrobial polymer, hereby referred to as “3-QAC-Hex”, has the structure shown below:

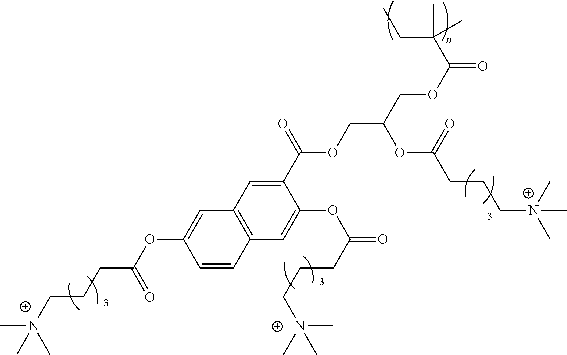

The antimicrobial polymer may be characterized in some respects by its ability to destroy microbes. As will be shown in the below Examples, the antimicrobial polymers of the present disclosure may be effective against a wide range of microbes. For instance, the antimicrobial polymers of the present disclosure may have minimum inhibitory concentrations (MIC) of between 1.0 μg/mL and 400 μg/mL, such as between 5.0 μg/mL and 300 μg/mL, such as between 10 μg/mL and 150 μg/mL.

The presently described antimicrobial polymer may be utilized in a variety of healthcare settings. In one embodiment, the polymer may be provided to a patient in a capsule form. Such a capsule form may allow for the antimicrobial polymers of the present disclosure to be administered to a patient in need of an antibiotic treatment.

In embodiments, the antimicrobial polymer of the present disclosure may be used as around the periphery of a wound dressing, or in combination with a semi-permanent or permanent access port into a patient's body. Thus, the antimicrobial polymer may be disposed on an exterior surface of a wound dressing, in injection device such as an intravenous (IV), arteriovenous (AV), catheter, orthopedic implant, or any suitable medical device.

In embodiments, the antimicrobial polymer of the present disclosure may be used in environments where sterility is to be maintained. For instance, the antimicrobial polymer of the present disclosure may be disposed on a sealing tape which can be used to seal biological specimens in a petri dish.

The present invention may be better understood with reference to the examples, set forth below.

### EXAMPLES

### Synthesis of Antimicrobial Polymers

To begin, polymeric backbones were synthesized. Three groups of polymer side chains were formed, with each group having either a four carbon or six carbon acyl halide. The four carbon chains, or Bu spacers, are discussed below. The six carbon chains, or Hex spacers, were synthesized in a similar fashion, except for the provision of a six carbon acyl halide where appropriate. FIGS. 3D, 3E, 3G, 4D, 4E, 4G, 5D, 5E and 5G correspond to the Hex spacer polymers.

FIGS. 3A-3F pertain to naphthoic acid polymers, FIGS. 4A-4F pertain to 6-hydroxynaphthoic acid polymers, and FIGS. 5A-5F pertain to 3,7-dihydroxnapthoic acid polymers.

### Synthesis of Poly(glycidyl methacrylate) (PGMA)

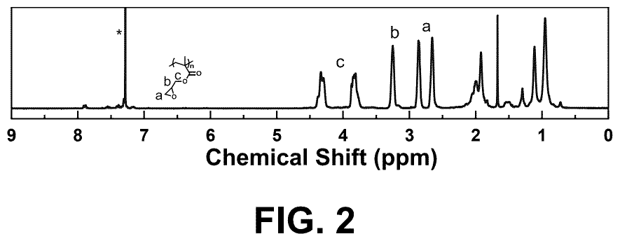

Glycidyl methacrylate was polymerized by using Reversible Addition-Fragmentation Chain Transfer (RAFT) polymerization technique. The molecular weight can be tuned by changing the molar ratios of monomer to chain transfer agent. Glycidyl methacrylate (4.74 mL, 33.30 mmol), cumyldithiobenzoate (CDB, 363.23 mg, 1.64 mmol), and azobisisobutyronitrile (AIBN, 43.79 mg, 0.27 mmol) were taken in a Schlenk tube and dissolved in DMF (4.00 mL). The mixture was applied with freeze-pump-thaw cycles three times and later protected with nitrogen. The solution was immersed into a preheated oil bath (set at 70° C.) and kept for a certain amount of time for desired molecular weight. The polymerization mixture was quenched with air and cooled down to room temperature. The DMF mixture was precipitated into excess methanol and solid polymer was collected. Later, the precipitated polymer was redissolved in DMF and precipitated again. This process was repeated 3 times. The obtained polymer was dried under vacuum (at 50° C.) to obtain a pink solid of the poly(glycidyl methacrylate) (PGMA). The obtained PGMA was confirmed by ¹H NMR, FT-IR, and GPC. The ¹H NMR (400 MHz, Chloroform-d) spectrum is shown in FIG. 2: δ=0.70-1.20 (m, 3H, —CH2-C—CH3), 1.85-2.06 (m, 2H, —CH3-CH2-C—CH3), 2.60 and 2.80 (s, 2H, —CH2-CH—CH2-O—), 3.20 (s, ¹H, —O—CH2-CH—CH2-O—), 3.63 and 4.45 (bs, 2H, —C═O—O—CH2-CH—O). GPC (THF): Mn=5.3×103 g/mol, Ð (Mw/Mn)=1.13.

### Synthesis of Naphthoic Acid-Containing Polymers

The synthesized PGMA (0.75 g, 5.28 mmol) and TBAB (91.43 mg, 0.28 mmol) were dissolved in 2-butanone (12.00 mL) and THF (3.00 mL) mixture. 2-Naphthoic acid (1.82 g, 10.56 mmol), 6-hydroxy-2-naphthoic acid (1.99 g, 10.56 mmol), or 3,7-dihydroxy-2-naphthoic acid (2.16 g, 10.56 mmol) was added to the polymer solution. The reaction mixture was placed in the preheated oil bath (set at 80° C.) and kept for 2 days under nitrogen. Afterward, the reaction mixture was cooled down to room temperature and precipitated into methanol twice. The naphthoic acid (NA) polymers were dried under vacuum at 50° C. Later, they were characterized by ¹H NMR, FT-IR, and GPC. Shown in FIG. 3A is the ¹H NMR spectrum of the NA polymer (400 MHz, DMSO-d6) spectrum: δ=0.70-1.10 (m, 3H, —CH2-C—CH3), 1.85-2.06 (m, 2H, —CH3-CH2-C—CH3), 3.74 and 4.45 (bs, 4H, —C═O—O—CH2-C(OH)—CH2-O—O═C—), 5.30-5.40 (s, ¹H, —O—CH2-CH(OH)—CH2-O), 7.26-7.63 (s, 2H, —CH— in the aromatic ring), 7.64-8.10 (d, 4H, —CH— in the aromatic ring), 8.72 (s, ¹H, —CH— in the aromatic ring).

6-hydroxy-2-naphthoic acid (HNA) and 3,7-dihydroxy-2-naphthoic acid (DHNA) containing polymers were precipitated in diethyl ether.

The ¹H NMR (400 MHz, DMSO-d6) spectrum of 6-hydroxy-2-naphthoic acid (HNA) is shown in FIG. 4A: δ=0.70-1.30 (m, 3H, —CH2-C—CH3), 1.85-1.96 (m, 2H, —CH2-C—CH3-), 3.74 and 4.45 (bs, 4H, —C═O—O—CH2-), 5.30-5.60 (s, ¹H, —CH2-O—CH—O—), 7.40-7.60 (s, 2H, —CH— in the aromatic ring), 7.64-8.10 (d, 3H, —CH— in the aromatic ring), 8.50 (s, ¹H, —CH— in the aromatic ring).

The ¹H NMR (400 MHz, DMSO-d6) spectrum of 3,7-dihydroxy-2-naphthoic acid (DHNA) containing polymers is shown in FIG. 5A: δ=0.70-1.11 (m, 3H, —CH2-C—CH3), 1.85-1.96 (m, 2H, —CH2-C—CH3-), 3.74 and 4.45 (bs, 4H, —C═O—O—CH2-), 5.30-5.60 (s, ¹H, —CH2-O—CH—O—), 6.92-7.33 (s, 3H, —CH— in the aromatic ring), 7.40-7.70 (d, ¹H, —CH— in the aromatic ring), 8.3 (s, ¹H, —CH— in the aromatic ring).

### Synthesis of Pendant Alkyl Bromide-Containing Polymers

NA polymer (0.40 g) was dissolved in 3.00 mL of DMF. The mixture was sealed and kept under the nitrogen atmosphere in an ice bath for 20 min. 4-Bromobutyryl chloride (2.60 mL, 22.5 mmol, 8.00 equiv.) was added to the ice-cold mixture dropwise. After reaching room temperature, the reaction mixture was placed in a 55° C. oil bath and kept stirring for 48 h. Later, the reaction mixture was cooled down to room temperature and precipitated into methanol 3 times. The obtained solid was dried under the vacuum at 50° C. The reaction was confirmed by ¹H NMR and FT-IR. The ¹H NMR of the NA polymer (400 MHz, Chloroform-d) spectrum is shown in FIG. 3B: δ=0.70-1.30 (m, 3H, —CH2-C—CH3-), 1.80-1.92 (m, 2H, —CH2-C—CH3-), 2.40-2.60 (t, 2H, —O—C═O—CH2-), 3.20-3.64 (m, 2H, —CH2-CH2-Br, 2H), 3.74 and 4.45 (bs, 4H, —C═O—O—CH2-), 5.30-5.60 (s, ¹H, —CH2-O—CH—O—), 7.26-7.63 (s, 2H, —CH— in the aromatic ring), 7.64-8.10 (d, 4H, —CH— in the aromatic ring), 8.72 (s, ¹H, —CH— in the aromatic ring). The elimination of OH groups on the NA polymer can be seen in the comparative FTIR before and after esterification of the pendant alkyl groups shown in FIG. 3F.

Similarly, HNA-containing polymers were modified by using 12 equiv. of 4-Bromobutyryl chloride. The ¹H NMR (400 MHz, Chloroform-d) spectrum is shown in FIG. 4B: δ=0.70-1.30 (m, 3H, —CH2-C—CH3-), 1.85-1.96 (m, 2H, —CH2-C—CH3-), 2.42-2.93 (t, 4H, —O—C═O—CH2-), 2.25-3.80 (m, 4H, —CH2-CH2-Br), 3.79 and 4.85 (bs, 4H, —C═O—O—CH2-), 5.30-5.60 (s, ¹H, —CH2-O—CH—O—), 7.10-7.30 (s, 2H, —CH— in the aromatic ring), 7.41-8.01 (d, 3H, —CH— in the aromatic ring), 8.45 (s, ¹H, —CH— in the aromatic ring). The elimination of OH groups on the NA polymer can be seen in the comparative FTIR before and after esterification of the pendant alkyl groups shown in FIG. 4F.

DHNA-containing polymers were modified by using 16 equiv. of 4-Bromobutyryl chloride. The ¹H NMR (400 MHz, Chloroform-d) spectrum is shown in FIG. 5B: δ=0.70-1.33 (m, 3H, —CH2-C—CH3-), 1.85-1.96 (m, 2H, —CH2-C—CH3-), 2.94-2.63 (t, 6H, —O—C═O—CH2-), 3.32-3.84 (m, 6H, —CH2-CH2-Br), 3.94 and 4.66 (bs, 4H, —C═O—O—CH2-), 5.30-5.60 (s, ¹H, —CH2-O—CH—O—), 7.43-7.93 (s, 4H, —CH— in the aromatic ring), 8.72 (s, ¹H, —CH— in the aromatic ring). GPC (THF): Mn=1.8×104 g/mol, Ð (Mw/Mn)=1.8. The elimination of OH groups on the NA polymer can be seen in the comparative FTIR before and after esterification of the pendant alkyl groups shown in FIG. 5F.

A second set of polymers having six carbon long chains were synthesized using, the same methods except instead of 4-Bromobutyryl chloride, 6-Bromohexanoyl chloride was used in the esterification step. The obtained polymers were characterized by ¹H NMR and FT-IR spectra. The ¹H NMR spectra can be found in FIGS. 3D, 4D and 5D. The FT-IR spectra can be seen in FIGS. 3F, 4F and 5F.

### Synthesis of Quaternary Ammonium Charged Naphthoic Acid-Containing Polymers

The NA polymer was dissolved in DMF and placed in a sealed ampoule. The ampoule was kept in the ice bath for 30 min. Then, an excess amount of 2.0 M trimethyl amine solution in THF was added. The reaction mixture was stirred at 55° C. overnight. The obtained precipitate was collected and dissolved in methanol and condensed. The condensed polymer solution was precipitated in THF. In the last step, the polymer was dissolved in water and dialyzed against 1 L water (3× times). The aqueous solution was transferred to a glass vial and freeze-dried. The obtained solid was dried under vacuum at 50° C. The ¹H NMR spectrum of the NA polymer (1-QAC-Bu) after quaternization may be seen in FIG. 3C (400 MHz, DMSO-d6): δ=0.70-1.30 (m, 3H, —CH2-C—CH3-), 1.85-2.05 (m, 2H, —CH2-C—CH3-), 2.38-2.62 (m, 2H, —CH2-CH2-N, 2H), 3.05-3.20 (m, 9H, —CH2-N—CH3-), 3.66 and 4.98 (bs, 4H, —C═O—O—CH2-), 5.30-5.60 (s, ¹H, —CH2-O—CH—O—), 7.26-7.63 (s, 2H, —CH— in the aromatic ring), 7.64-8.10 (d, 4H, —CH— in the aromatic ring), 8.72 (s, ¹H, —CH— in the aromatic ring.

HNA polymer was similarly quaternized The ¹H NMR spectrum of the HNA polymer (2-QAC-Bu) after quaternization may be seen in FIG. 4C (400 MHz, DMSO-d6): δ=0.70-1.30 (m, 3H, —CH2-C—CH3-), 1.60-2.15 (m, 2H, —CH2-C—CH3-), 2.18-2.52 (m, 2H, —CH2-CH2-N, 4H), 2.90-3.10 (m, 18H, —CH2-N—CH3-), 3.66 and 4.98 (bs, 4H, —C═O—O—CH2-), 5.30-5.60 (s, ¹H, —CH2-O—CH—O—), 7.26-7.63 (s, 2H, —CH— in the aromatic ring), 7.64-8.10 (d, 4H, —CH— in the aromatic ring), 8.72 (s, ¹H, —CH— in the aromatic ring).

DHNA polymer was similarly quaternized. The ¹H NMR spectrum of the DHNA polymer (3-QAC-Bu) after quaternization may be seen in FIG. 5C (400 MHz, DMSO-d6): δ=□0.70-1.20 (m, 3H, —CH2-C—CH3-), 1.65-2.10 (m, 2H, —CH2-C—CH3-), 2.18-2.42 (m, 6H, —CH2-CH2-N, 2H), 2.90-3.10 (m, 27H, —CH2-N—CH3-),3.66 and 4.98 (bs, 4H, —C═O—O—CH2-), 5.30-5.60 (s, ¹H, —CH2-O—CH—O—), 6.92-7.33 (s, 3H, —CH— in the aromatic ring), 7.40-7.70 (d, ¹H, —CH— in the aromatic ring), 8.3 (s, ¹H, —CH— in the aromatic ring).

The ¹H NMR spectra of the six carbon long chains can be seen in FIGS. 3E (1-QAC-Hex, derived from the NA polymer), 4E (2-QAC-Hex, derived from the HNA polymer), and 5E (3-QAC-Hex, derived from the DHNA polymer).

### Measurements of Antimicrobial Activity

Except 1-QAC-Bu, all Hex and Bu spacer bearing single, double, and triple-charged cationic polymers are readily soluble in water. Escherichia coli (E. coli, ATCC-25922), Pseudomonas aeruginosa (P. aeruginosa, ATCC-27853), Klebsiella pneumoniae (K. pneumoniae, ATCC-13883), Klebsiella oxytoca (K. oxytoca, ATCC-49131), Staphylococcus aureus (S. aureus, ATCC-29213), Acinetobacter baumannii (A. baumanii, ATCC-19606) multi-drug resistant (MDR) P. aeruginosa (MDR-PA, ATCC BAA-3107), multi-drug resistant Klebsiella pneumoniae (MDR-KP, ATCC BAA-2473), and multi-drug resistant E. coli (MDR-EC, ATCC BAA-197) were purchased from ATCC. Bacteria were first streaked on Tryptic Soy agar (TSA) plates and incubated at 37° C. for 24 h. A single colony was inoculated in 3 mL Tryptic Soy broth (TSB) at 37° C. for 6 h, shaking at 190 rpm to reach the mid-log phase. All bacteria were adjusted to an optical density of about 0.5 McFarland Standard (OD600=0.70) for further use.

### Minimum Inhibitory Concentration (MIC) Assay

The minimum inhibitory concentration (MIC) test was performed. MIC is the lowest concentration of an antibacterial agent expressed in mg/L (μg/mL) which, under strictly controlled in vitro conditions, completely prevents visible growth of the test strain of an organism.

Different concentrations of 20 μL of the aqueous solution of 1-QAC-Bu, 2-QAC-Bu, 3-QAC-Bu, 1-QAC-Hex, 1-QAC-Hex and 1-QAC-Hex by using the microdilution technique were added to 96-well plates. Then, 180 μL bacterial solution (2-7×105 CFU/mL) was transferred to each well. The bacterial solutions and media without the above polymers were used as the positive control, and only media without compounds and bacteria was used as the negative control. The prepared 96-well plate was placed into an incubator at 37° C. shaking at 190 rpm overnight. The bacterial growth was measured by reading OD600 values using a spectrophotometer. The MIC was recorded as the lowest concentration of the compound that completely inhibits bacterial growth. All tests were performed three times, each in triplicate measurements.

### Minimum Bactericidal Concentration (MBC) Assay

A. baumannii, MDR-E. coli, MDR-P. aeruginosa and MDR-K. pneumoniae bacteria were treated with 1-QAC-Hex and 3-QAC-Hex. Mid-log phase (˜108 CFU/mL) bacterial cell cultures were suspended to ˜105 CFU/mL in media. 175 μL of this bacterial suspension was mixed with 25 μL of MIC/2, MIC, and 2×MIC of the corresponding compound followed by incubation at 37° C. overnight. As a negative control, the same volume (25 μL) of water was used instead of any antibacterial compounds. Samples of 20 μL of each concentration were then 10-fold serially diluted in 1× phosphate buffer system (PBS). From these dilutions, 20 μL was spot plated on a tryptic agar plate and incubated for 24 h at 37° C. for counting viable cells. These experiments were performed in duplicate.

### Time-Kill Kinetics Against Planktonic Bacterial Cells

The kinetic experiment was performed similarly as described in the minimum bactericidal concentration assay. A. baumannii, MDR-E. coli, MDR-P. aeruginosa and MDR-K. pneumoniae bacteria were treated with 1-QAC-Hex and 3-QAC-Hex. Mid-log phase (˜108 CFU/mL) bacterial cell cultures were suspended to ˜105 CFU/mL in media. 175 μL of this bacterial suspension was mixed with 25 μL of MIC/2, MIC, and 2×MIC of the corresponding compounds followed by incubation at 37° C. overnight. As a negative control, 25 μL of water was used instead of any antibacterial compounds. 20 μL samples of solution from each concentration were then 10-fold serially diluted in 1×PBS at various time points such as 0, 1, 2, 4, 8, 16, 20, and 24 h. From these dilutions, 20 μL aliquots were spot plated on a tryptic agar plate and incubated for 24 h at 37° C. After incubation, viable colonies were counted on the plates. 50 CFU/mL is the detection limit of this experiment. These experiments were performed in duplicate.

### Time-Kill Kinetics Against Dormant Cells

This experiment was performed for 1-QAC-Hex against MDR-P. aeruginosa. Dormant cells are prepared as followings: mid-log phase (˜108 CFU/mL) bacterial cell culture was suspended to ˜105 CFU/mL in nutrient broth (NB) and incubated at 37° C. with 190 rpm shaking for 16 h. After incubation, the bacterial cell culture was centrifuged at 3500×g for 5 min and the supernatant was removed. The bacterial cell pellet was then resuspended in 1×PBS. The bacterial cell density was adjusted by 0.5 McFarland Standard (OD600=0.70). The dormant cells were then used for the kinetic experiment. The later steps were the same as time-kill kinetics against planktonic bacterial cells. These experiments were performed in duplicate.

### Biofilm Eradication Assay

18 mm glass coverslips were sterilized with flame after rinsing with ethanol. The bacterial biofilms were grown by following the previously published protocol. After the preparation of mid-log grown MDR-P. aeruginosa bacteria in TSB media, bacteria concentration was adjusted in NB with 1% (w/v) glucose and 1% (w/v) NaCl supplements to ˜105 CFU/mL. 2 mL of the diluted bacterial cell culture was used to fill the 6-well plate and 18 mm sterilized glass coverslips were added to each well. The plate was incubated at 37° C. for 72 h. After 72 h, 1×PBS (pH=7.4) was used to wash away unadhered planktonic bacteria. The carefully washed coverslips containing surface-attached biofilms were placed into a new 6-well sterile plate. Later, various concentration of 1-QAC-Hex (2 mL) was added into the wells and incubated for 24 h. Polymyxin-B was used as a control antibiotic. As a negative control, 2 mL of media was added without compound. The following three experiments were adjusted in the further steps as follows.

### Quantification Through Bacterial Cell Viability

Once again, coverslips were washed with 1×PBS and transferred to another new, sterile plate. Afterward, 2 mL of trypsin-EDTA solution (diluted in saline at a 1:10 ratio) was added to each well and incubated for 15 min. Samples of 20 μL of each concentration were then 10-fold serially diluted in 1×PBS. From these dilutions, 20 μL was spot plated on a tryptic Soy agar plate and incubated for 24 h at 37° C. for counting viable colony forming units released from the biofilms. These experiments were performed in duplicate.

### Crystal Violet Staining of Biofilm Biomass

Once again, coverslips were washed with 1×PBS and transferred to another new, sterile plate. Afterward, 0.1% of crystal violet (1 mL) dye was carefully added and left for 10 min for proper dying. The crystal violet stained coverslips were taken out and the last wash with 1×PBS was applied to remove excess crystal violet. The crystal violet stained biofilm containing coverslip was imaged and immediately dissolved with 95% ethanol. The absorbance of crystal violet containing ethanol was recorded at 520 nm. The absorbance of crystal violet dye was interpreted to correlate with the amount of biomass.

### Fluorescence Microscopy of Biofilm Disruption

Coverslips were washed with 1×PBS and left to dry for 2 min. MDR-P. aeruginosa biofilm was simultaneously stained with equal volumes of (5 μL) SYTO 9 (60 μM) and PI (15 μM) and was placed on the slide immediately followed by incubation in darkness for 15 min. The glass coverslips images on the slide were captured with a Zeiss LSM 410 Confocal Laser Scanning Microscope in 40× magnification. Further, the images were processed by Image J software.

### Scanning Electron Microscopy

Briefly, 1 mL of ˜106 CFU/mL mid-log phase cells of MDR P. aeruginosa (ATCC BAA-3107) was treated with 3-QAC-Hex (64 μ/mL) in Muller Hinton broth (MHB) in a 12 well plate. In case of control, bacterial cells were not treated with the compound. Further, two sterilized coverslips (tissue culture treated and 13 mm diameter) were added into each 1 mL bacterial suspension (in 12 well) immediately in case of treated and untreated samples and incubated for 16 h at 37° C. under static condition. Afterward, the coverslips were taken out from the wells and processed for SEM imaging. First, the coverslips (bearing the bacterial cells) were incubated with 2.5% glutaraldehyde solution in 0.1 M sodium cacodylate buffer for 2 h at 4° C. Next, the same coverslips were incubated in 1% aqueous solution of osmium tetroxide for another hour at 4° C. and they were further washed with 0.1 M sodium cacodylate buffer to remove trace amount of toxic osmium tetroxide. Next, the coverslips were gradually incubated with 50%, 70%, 80%, 95% and 100% ethanol and each incubation step was for 10 min at room temperature. Finally, the coverslips (containing fixed and dehydrated bacterial cells) were dried in a critical point chamber, then coated with gold using a Denton Desk II sputter coater for 60 s, and images were captured using Tescan Vega-3 SBU SEM instrument.

### Resistance Study

The resistance development study was performed following the protocol published earlier. The experiment was performed for 1-QAC-Hex and 3-QAC-Hex against MDR-P. aeruginosa and MDR-K. pneumoniae, respectively. To compare the result, the last-resort gram-negative antibiotics, Polymyxin-B, and carbapenem type, Imipenem, were used in the study. According to the protocol elaborated for the MIC assay above, the MIC values of compounds and antibiotics were determined against corresponding bacteria overnight. The next day, a new MIC assay was performed where the bacterial suspension was prepared from the bacteria grown at the sub-MIC (MIC/2) of compound concentration of the first-day experiment. The next 14 continuous passages were repeated as described above. The resistance development was calculated as the fold increase of MIC by dividing MIC to the initial MIC values of every passage and then plotted against the passage number. These experiments were performed in triplicate.

### Results

The results of the above experimental method are discussed below.

The synthesis of naphthoic acid-based cationic polymers was carried out through four-step reactions starting from glycidyl methacrylate (GMA). Given that many AMPs are oligopeptides typically comprising of 10-60 amino acid residues, poly(glycidyl methacrylate) (PGMA) with about 30 repeat units was synthesized via reversible addition-fragmentation chain transfer (RAFT) polymerization by using 2-phenyl-2-propyl benzodithioate as a chain transfer agent. This homopolymer has a molecular weight of 5000 g/mol and low dispersity (Ð=˜1.2) Multiple charged polymers were obtained by post-polymerization modification as shown in Scheme 1. First, 2-Naphthoic acid (NA), 6-hydroxy naphthoic acid (HNA), and 3,7-dihydroxy naphthoic acid (DHNA) were introduced into polymers through ring opening of the epoxy group with the retention of hydroxyl group. ¹H NMR spectra (FIG. 2) confirmed the presence of phenyl rings at ˜7-9 ppm and the disappearance of epoxy groups at 3.2 ppm. Hydroxyl group(s) were used for further esterification with 4-Bromobutyryl bromide. The disappearance of absorption in the IR spectrum for the hydroxyl group after esterification with acyl chloride confirmed the full conversion of hydroxyl groups. Also, the NMR spectrum showed the —CH2 group next to —Br at ˜3.3-3.6 ppm. In the final step, bromide group(s) were quaternized with trimethylamine to obtain amphiphilic homopolymers. Using NA, HNA, and DHNA, single (1-QAC-Bu), double (2-QAC-Bu), and triple (3-QAC-Bu) charged naphthoic acid-based PGMA homopolymers were obtained, all of which have a four-carbon ester linker (designated as Bu) between the hydrophobic fused ring and cationic charge. After quaternization, the appearance of an intense peak at ˜3.0 ppm for three methyl groups was observed by ¹H NMR. Quaternization was also confirmed with changed solubility, all cationic-charged homopolymers were soluble in water except the single charged polymer (1-QAC-Bu). The synthesized polymers were characterized by ¹H NMR, FT-IR, and GPC and presented in supplementary information.

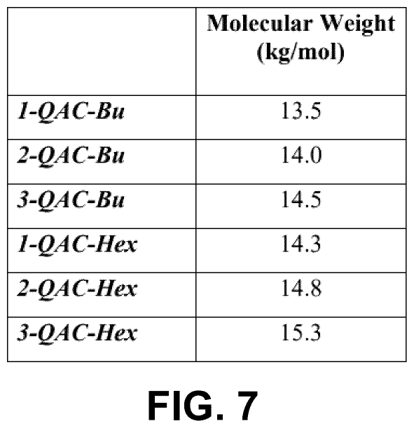

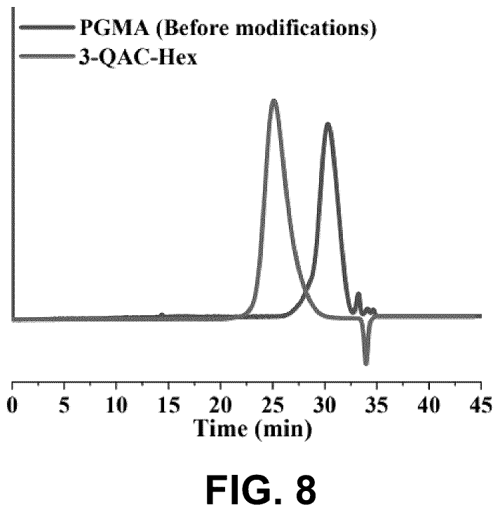

To examine the effect of space linker between the hydrophobic fused ring and cationic charge on antimicrobial activity, another set of polymers were synthesized with a six-carbon ester linker (designated as Hex) between naphthoic and QAC groups. Polymers were labeled with the number of positive charges, and the structure of linkers, as shown in FIG. 4E (i.e., 2-QAC-Hex is 6-hydroxy-2-naphthoic acid modified PGMA with a six-carbon linker). A summary of six antimicrobial polymers is shown in FIG. 6. Their molecular weights and gel elution times are shown in FIGS. 7 and 8 respectively.

### Antimicrobial Activity

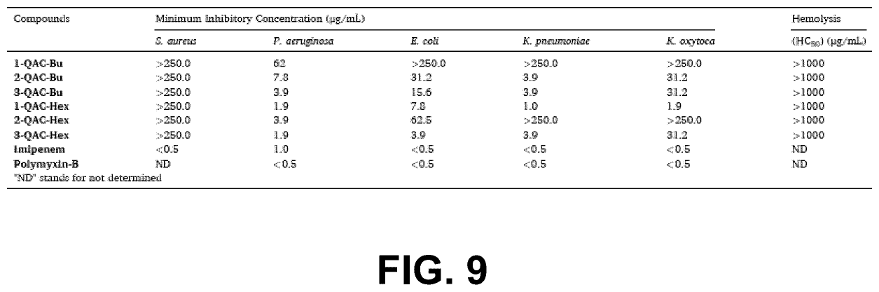

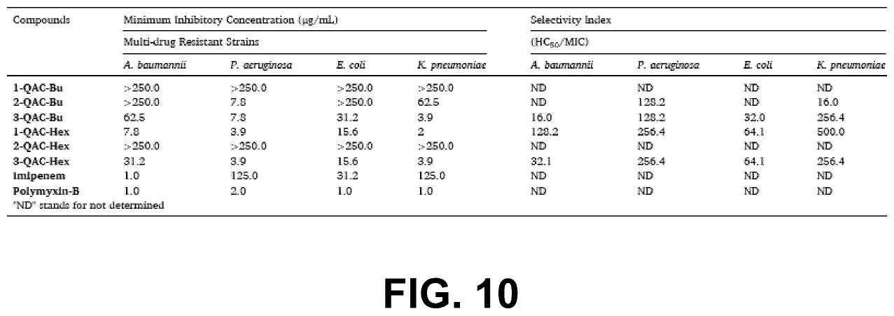

Initially, the antimicrobial activity of facial amphiphilic and multiple charged naphthoic acid-based PGMA polymers was determined against gram-positive (S. aureus) and gram-negative (E. coli, P. aeruginosa, and K. pneumoniae) strains. It was carried out through a broth microdilution method following a previously established protocol adopted by the Clinical Laboratory Standards Institute (CLSI) and reported as minimum inhibitory concentration (MIC). Polymers were tested in a concentration ranging from 250 μg/mL to 0.5 μg/mL. Imipenem and polymyxin-B, the last-resort antibiotics, were used as positive controls. The details of the antibacterial activities of polymers and control antibiotics are listed in the table shown in FIGS. 9 and 10.

While none of the polymers show any activity towards S. aureus, most of them display potent antimicrobial activity against gram-negative strains. The most active polymer with a Bu spacer against P. aeruginosa was the triple charged polymer (3-QAC-Bu) with a MIC value of 3.9 μg/mL, followed by double charged polymer (2-QAC-Bu) and single charged polymer (1-QAC-Bu) at 7.8 and 62.5 μg/mL, respectively. Even though the triple charged polymer displayed an appreciable antibacterial activity, the single-charged polymer had poor antibacterial activity. Similarly, the MIC value increased as the number of charges decreased: from 15.6 to >250 μg/mL against E. coli, from 3.9 to >250 μg/mL against K. pneumoniae, from 31.2 to >250 μg/mL against K. oxytoca. Since the hydrophobic pendant alkyl chain (Hex) of polymers (bicyclic architecture) is similar among three different naphthoic structures, the results suggest that antibacterial activity may be strongly dependent on hydrophilic (single, double, and triple) charged groups. This result may be essentially dictated by facial amphiphilic index (FAI) (charges vs. number of rings), varying from 1/2 to 2/2 to 3/2. A similar charge-dependent behavior was observed in our previous work and reported by the other groups in the literature.

To investigate the effect of a spacer between hydrophobic rings and cationic moieties, a longer aliphatic linker (Hex) was introduced to obtain the second set of polymers. Compared with the Bu linker, the single charged polymer (1-QAC-Hex) has a significant improvement of MIC values at 1.9, 7.8, 1.0, and 1.9 μg/mL against P. aeruginosa, E. coli, K. pneumoniae, and K. oxytoca respectively. The triple charged polymer (3-QAC-Hex) displayed similar behaviors with some MIC values decreased to 4-fold. However, the double charged polymer (2-QAC-Hex) showed poor antibacterial activity against all strains except P. aeruginosa. This positive effect may be related to the increased distance between the hydrophobic group and the cationic charge. While the increased charge density may provide better electrostatic interactions with the negatively charged bacterial membrane, the longer chain may contribute to the deeper penetration into the hydrophobic membrane interior which is known as the snorkeling effect reported in the literature. Overall, a general extraction from the results suggests that a delicate balance in amphiphilicity may be the key factor to achieving good antibacterial activity.

After discovering the selective antibacterial activity of these polymers against gram-negative strains, the testing was extended to MDR strains. The World Health Organization (WHO)-designated ESKAPE strains are a priority list of six MDR pathogens, including four gram-negative bacteria, Klebsiella pneumoniae, Acinetobacter baumannii, Pseudomonas aeruginosa, and Enterobacter species. The MIC values against the four gram-negative MDR strains are listed in the table shown in FIGS. 9 and 10. Polymers 1-QAC-Bu, 3-QAC-Bu, 1-QAC-Hex, and 3-QAC-Hex exhibited appreciable antibacterial activities. Comparatively, 1-QAC-Hex and 3-QAC-Hex were considered to be lead polymers, as they displayed better activities with MIC values of 7.8-31.2, 3.9, 15.6, and 1.0-3.9 μg/mL against A. baumannii, MDR-P. aeruginosa, MDR-E. coli, and MDR-K. pneumoniae, respectively.

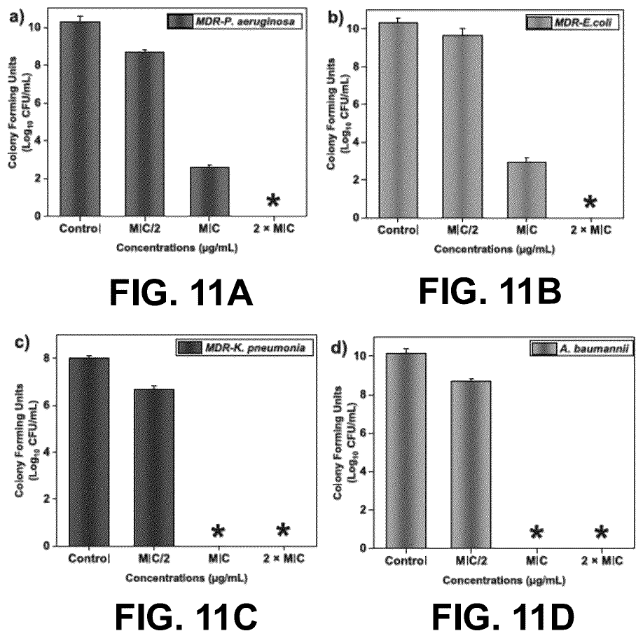

While polymers of the present invention may inhibit bacterial growth, there could remain surviving with a negligible order of colony-forming units. Thus, minimum bactericidal concentration (MBC) was also evaluated. All MBC results of two lead polymers, 3-QAC-Hex (FIGS. 11A and 11B) and 1-QAC-Hex (FIGS. 11C and 11D), against MDR pathogens are given in FIG. 11. While MDR-P. aeruginosa and MDR-E. coli were completely killed at 2×MIC after treating with 3-QAC-Hex, MDR-K. pneumoniae and A. baumannii were irreversibly inhibited at one MIC after being exposed to 1-QAC-Hex. It is noteworthy to mention that MDR-P. aeruginosa and MDR-K. pneumoniae are less susceptible to antimicrobial peptide (AMP) mimics and antibiotics since both pathogens have low permeability of their cell envelopes, which dictate the intrinsic and acquired AMR behavior.

### Hemolytic Activity, Toxicity, and Selectivity Index

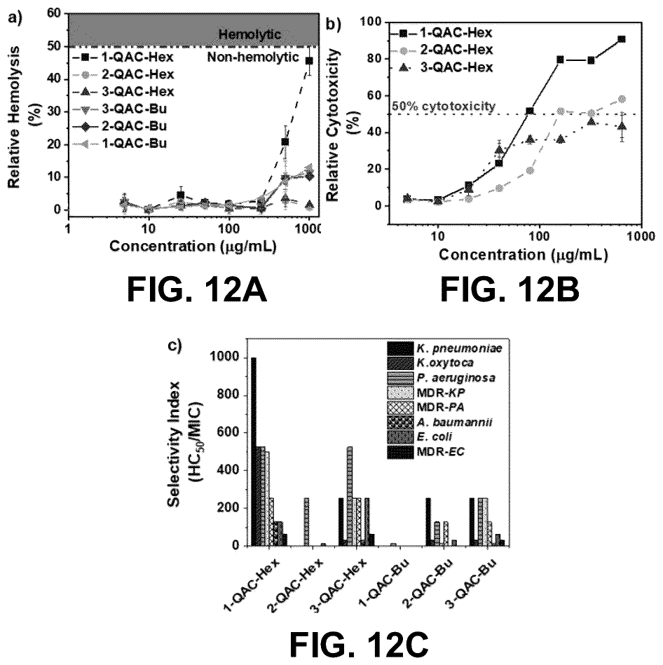

To evaluate the biocompatibility of these antimicrobial polymers, hemolysis of mouse red blood cells (RBCs) and cytotoxicity against human embryonic kidney cells were conducted and summarized in FIG. 12. Hemolysis was given as HC50 where the concentration of a polymer causes 50% lysis of RBCs. As a positive control, Triton-X 100 was used, which induced 100% lysis of RBCs. RBC in PBS was used as the negative control to subtract any background signals. All polymers have HC50 significantly over 1000 μg/mL (FIG. 9).

A lactate dehydrogenase (LDH) toxicity assay was conducted as stated in the follow paragraph. Human embryonic kidney cells (HEK-293) were seeded in a 96-cell plate for 24 hours. At the time of testing, cell confluency should be between 50% and 80%. The medium was changed to Delgado's Modified Eagle Medium (DMEM) w/5% fetal bovine serum (FBS) (total volume is 150 μL). Compounds were added with indicated concentrations to the wells and incubated at 37° C. for varied time. Cells incubated with lysis buffer from the kit (BioLegend LDH-Cytox Assay kit, cat #426401) were used as the maximal toxicity controls. After incubation, 100 μL supernatant S6 from each well was transferred to a new 96-well plate and 100 μL of LDH assay working solution was added according to the provided instruction. The absorbance was measured at 490 nm. Wells with medium only served as background controls. Relative toxicity was calculated according to: (Sample OD−background)/(Max lysis OD−background) The results are given with the standard error bars of mean. These experiments were performed in triplicate.

In vitro cytotoxicity of Hex-bearing polymers was tested against human embryonic kidney cells (HEK-293) through LDH assay at a concentration range of 5-640 μg/mL. Dose-dependent curves for 3-QAC-Hex, 2-QAC-Hex, and 1-QAC-Hex are given in FIG. 12B. The concentration that kills 50% of the corresponding cells was determined as IC50. The single and double-charged polymers, 1-QAC-Hex, and 2-QAC-Hex displayed IC50 values at 78 μg/mL and 200 μg/mL, respectively. The increased charge density reduced the toxicity. The most active compound, triple-charged polymer (3-QAC-Hex), had an IC50 over 640 μg/mL.

A suitable antimicrobial agent should display selective antimicrobial characteristics towards pathogenic microbes over mammalian cells. The selectivity of polymers is defined as the ratio between the concentrations leading to 50% hemolysis and the minimum inhibitory concentration for bacterial growth (Selectivity index (SI)=HC50/MIC). SI results are summarized in FIGS. 12A-C. Larger SI values indicate selective killing of pathogens over mammalian cells. Since all polymers of the present disclosure have HC50 over 1000 μg/mL, the selectivity index depends on the MICs of QAC polymers. The bar plot in FIG. 12C shows the selectivity index of the polymers of the present disclosure against bacterial strains. The species at the top of the legend corresponds to the leftmost bar within a specific treatment group; the selectivity index of 1-QAC-Hex against bacterial species is shown in the order of K. pneumoniae, K. oxytoca, P. aeruginosa, MDR-K pneumoniae, MDR-P. aeruginosa, A. baumannii, E. coli and MDR-E. coli from left to right. The same ordering applies to the remaining antimicrobial polymers, 2-QAC-Hex, 3-QAC-Hex, 1-QAC-Bu, 2-QAC-Bu and 3-QAC-Bu. In general, better selectivity was obtained for polymers containing a Hex linker. For 3-QAC-Hex and 1-QAC-Hex, the selectivity index is in the range from 32.1 to 500.0 or higher, which manifests excellent selectivity toward bacterial membranes over mammalian membranes.

### Bactericidal Kinetics Against Planktonic Bacteria

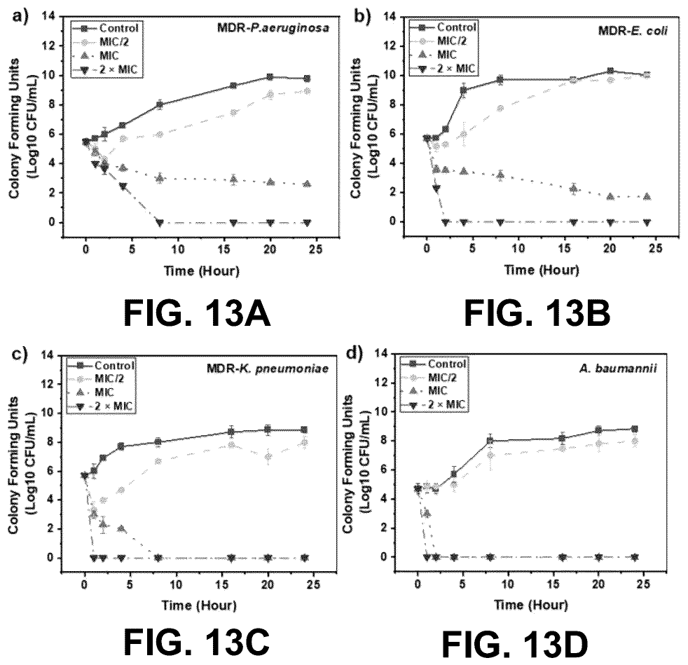

Time-killing kinetic assay of two lead polymers was employed. Four opportunistic bacteria were tested, and the results are given in FIG. 13A-13D. 3-QAC-Hex was tested against MDR-E. coli and MDR-P. aeruginosa, while 1-QAC-Hex was used for MDR-K. pneumoniae and MDR-A. baumannii. A gradual reduction of bacterial population was observed when the concentration reached a certain level. After treatment with 3-QAC-Hex, a complete killing (˜5 Log CFU/mL reduction) was achieved after 2 hours at 31.2 μg/mL (2×MIC) against MDR-E. coli and after 8 h at 3.8 μg/mL (2×MIC) against MDR-P. aeruginosa while the control increased to ˜7-8 Log CFU/mL at similar time points.

In comparison, 1-QAC-Hex showed a ˜5 Log CFU/mL reduction of MDR-K. pneumoniae just one hour after treatment with 2.0 μg/mL (2×MIC). Even at the MIC (1.0 μg/mL), this polymer also effectively killed all pathogens after 8 hours of treatment. 1-QAC-Hex displayed similar kinetics against MDR-A. baumannii to MDR-K. pneumoniae at both MIC and 2×MIC treatments. Those results confirmed that naphthoic acid-based charged polymers may have a rapid killing ability against MDR bacteria.

### Biofilm Eradication

As naphthoic acid-based polymers may exhibit excellent efficacy against MDR bacterial strains, the antibiofilm ability of naphthoic acid-based polymers was then evaluated. Biofilms contain highly refractory bacterial cells that are encased by an extracellular polymeric matrix (EPS), which protects metabolically inactive and dormant cells. This matrix not only isolates the bacterial cells but also works as a strong barrier to prevent the penetration of antibiotics. Once this matrix is formed, it paves the way for anchoring more bacterial cells and can drive infections to be long-lasting and untreatable. The biofilm eradication ability of a lead polymer 3-QAC-Hex was tested against MDR-P. aeruginosa, which accounts for complicated and chronic infections and causes a major and serious threat in health care settings.

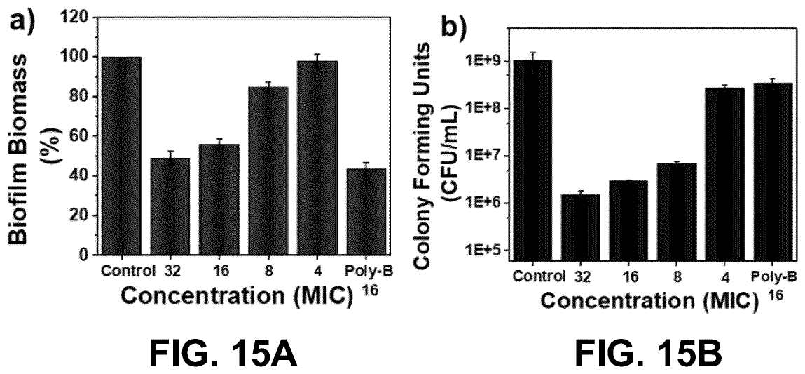

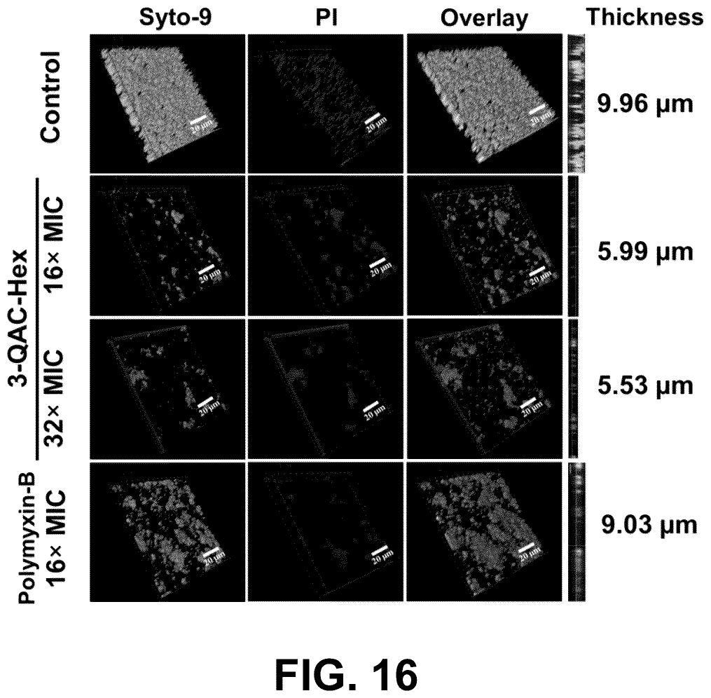

First, crystal violet staining was used to measure biofilm biomass upon treatment, (FIG. 15A). The biofilm biomass was reduced ˜40% and ˜50% at 16×MIC and 32×MIC, respectively. Next bacterial cell viability was analyzed by counting live cells within the biofilm and in dispersed cells. The 16×MIC and 32×MIC of 3-QAC-Hex were able to achieve killing of 99.85% and 99.71%, respectively, encased bacteria in the biofilm matrix. In another word, the bacterial burden was reduced by ˜3 Log as shown in FIG. 15B. Even though 16×MIC of polymyxin-B can reduce the biofilm biomass to a similar level to 16×MIC polymer (FIG. 15A), it was unable to kill the viable bacteria protected within the biofilm, in contrast to the polymer of the present invention that showed significant killings (FIGS. 15B and 16).

Lastly, the biofilm eradication was visualized by using confocal laser microscopy and biomass reduction was quantified by Z-stack analysis. Polymyxin-B 16×MIC was tested against 16×MIC and 32×MIC 3-QAC-Hex polymers. The polymyxin-B 16×MIC sample contained a large amount of live cells after treatment. The 3-QAC-Hex S samples contained largely dead cells. The thickness of biofilm was measured as 9.96 μm for control and 9.03 μm for 16×MIC treated polymyxin-B, while only 5.99 μm and 5.53 μm for 16×MIC and 32×MIC of 3-QAC-Hex treated samples. Overall, it can be concluded that 3-QAC-Hex showed promising anti-biofilm activity and might be used in clinical settings to combat refractory MDR-P. aeruginosa.

### Killing Ability Against Dormant Bacteria

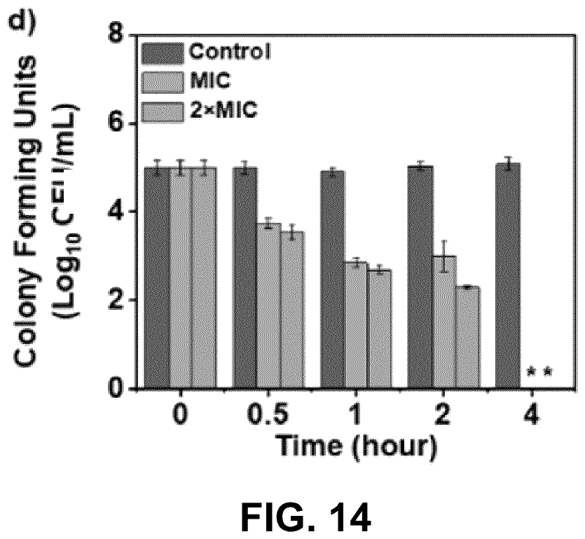

In addition to biofilms, 3-QAC-Hex was evaluated against dormant cells of MDR-P. aeruginosa. Dormant cells are metabolically inactive and stress-tolerant cells, and also may be known as excessive manipulators of antibiotic treatments. Dormant cells can develop within biofilms or form in the presence of a lethal stress environment and can be responsible for antimicrobial resistance. Even though those cells are a transiently established subpopulation of bacterial cells, they can ensure serious long-lasting infections and account for intractable treatment since their mechanisms of formation and growth are not well understood. Therefore, evaluating the killing ability of promising antimicrobial and antibiofilm agents against dormant cells is significantly important. The killing ability of the lead polymer 3-QAC-Hex was tested. 3-QAC-Hex exhibited complete killing against dormant cells at MIC (3.9 μg/mL). Later, the time-killing kinetics was carried out, the results demonstrated that both MIC and 2×MIC provided ˜2 Log reduction after one hour and complete killing after four hours as shown in FIG. 14. The bars in FIG. 14 are presented in the order of the control, minimum inhibitory concentration and 2× minimum inhibitory concentration from left to right for each time increment.

### Bacterial Membrane Perturbation

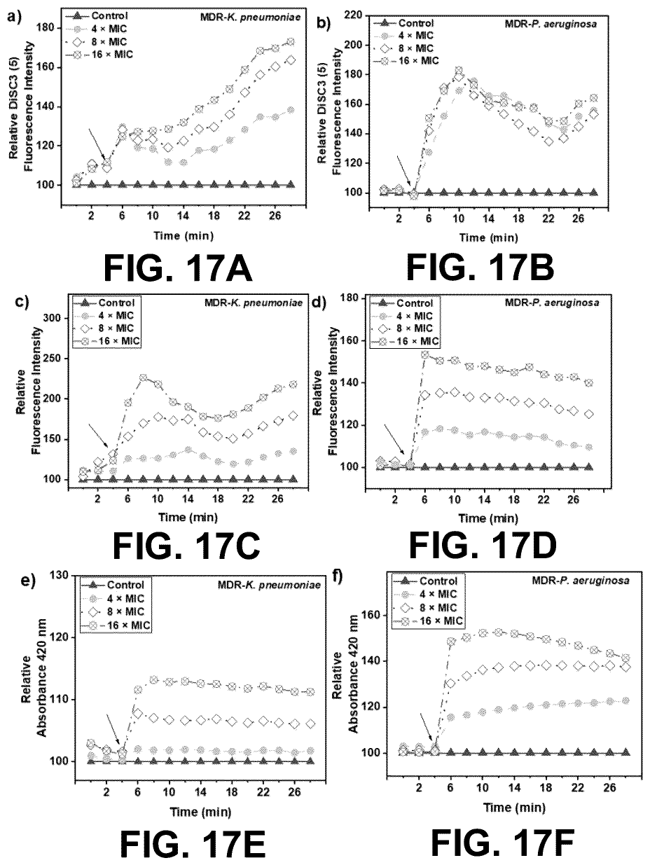

The ability of 1-QAC-Hex and 3-QAC-Hex to disrupt membranes was investigated against planktonic cells of bacteria (FIG. 17). Since MDR-P. aeruginosa and MDR-K. pneumoniae are gram-negative strains, both inner and outer membrane permeabilization experiments were performed along with membrane depolarization. 3,3′-Dipropylthiadicarbocyanine iodide (DiSC3(5)) was used as a dye for depolarization test. The fluorescence intensity increased immediately after addition of 4×, 8×, 16×MIC of 3-QAC-Hex and 1-QAC-Hex (at 4 min as showed with arrows), indicating the loss of membrane potential and entrance of dye into the cytoplasmic membrane (FIGS. 17A and 17A). 1-N-phenylnaphthylamine (NPN) was used to test permeabilization of the outer membrane, which is composed of glycolipids. If the membrane was destroyed, NPN would interact with these lipids, resulting in prominent fluorescence (emission at 420 nm). After exposure of 4×, 8×, 16×MIC of 3-QAC-Hex and 1-QAC-Hex (at 4 min as showed with arrows), the quick increase of fluorescence intensity was observed against both MDR strains (FIGS. 17C and 17D). The fluorescence intensity was greater at higher concentrations of polymers, indicating dose-dependent membrane permeabilization. Lastly, the inner membrane disruption was monitored by adding ortho-Nitrophenyl-β-galactoside (ONPG), which is hydrolyzed by the cytoplasmic β-galactosidase enzyme. This interaction would result in the release of galactose and ortho-nitrophenol, which has an absorbance at 420 nm. With the addition of 4×, 8×, 16×MIC of 3-QAC-Hex and 1-QAC-Hex (at 4 min as showed with arrows), significant absorbance increments were monitored with time (FIGS. 17E and 17F). The results clearly showed that both inner and outer layers of bacterial strains were disrupted after being exposed to the 1-QAC-Hex and 3-QAC-Hex.

### Bacterial Cell Morphology Via SEM

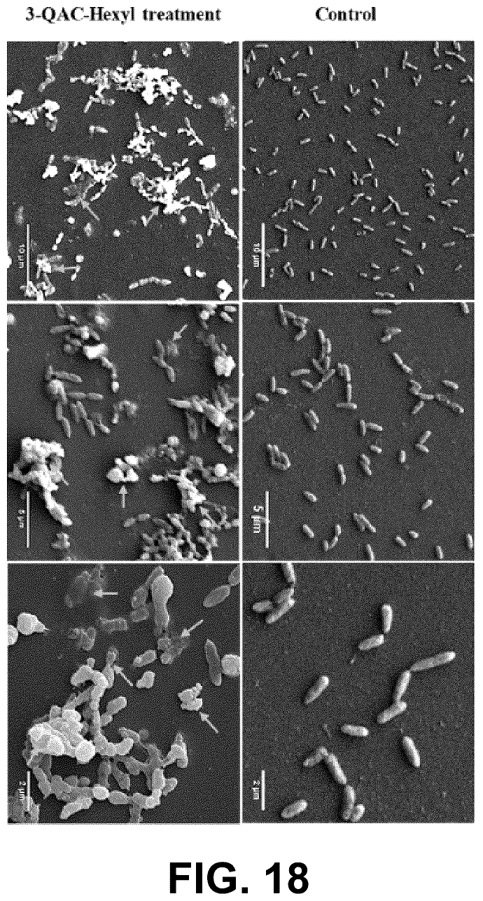

Further, the morphology of bacterial cells was monitored by scanning electron microscopy (SEM) (FIG. 18). The mid-log phase cells of MDR-P. aeruginosa were treated with 3-QAC-Hex (64 μg/mL) in Muller Hinton broth for 16 h. The untreated cells (control) displayed a healthy rod-shaped cellular morphology. The polymer treated bacterial cells revealed the presence of a high concentration of cell debris, and compromised cells with holes and blebs are indicated by arrows in the left column of FIG. 18. These results demonstrated the visual changes of bacterial membrane damage due to the membrane active nature of this class of cationic polymers of the present disclosure. Taken together, this study indicated that the microbial membrane is the primary target of the 3-QAC-Hex polymer.

### Resistance Study

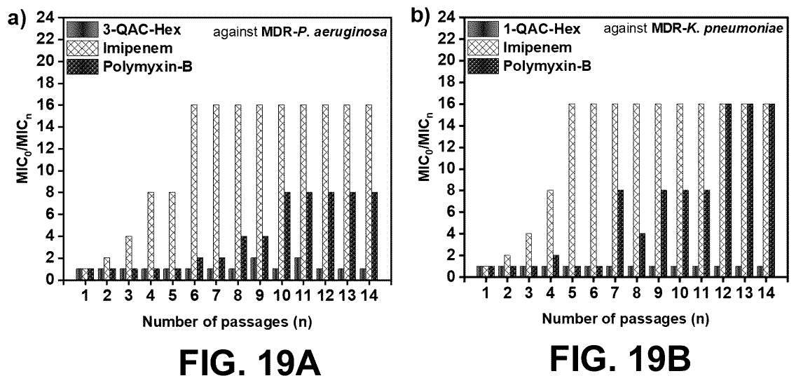

MDR bacteria employ various strategies inherently or adaptively to build up resistance, which becomes a serious health concern. The antimicrobial resistance is identified as greater than a 4-fold increase in the initial MIC when exposing the organism to the same antimicrobial agent. To evaluate the potential antimicrobial resistance of 1-QAC-Hex and 3-QAC-Hex against MDR-P. aeruginosa and MDR-K. pneumoniae, an antimicrobial resistance study was conducted. Bacteria were exposed to continuous serial passaging at the sub-MIC level over 2 weeks. Antibiotics imipenem and polymyxin-B were used as controls. MDR-P. aeruginosa obtained resistance to both antibiotics with the change of MICs by 16-fold and 8-fold, respectively (FIG. 19A, wherein the bars for each passage is in the order of the antimicrobial polymer, imipenem and polymyxin-B). Similarly, MICs of imipenem and polymyxin-B evolved to a 16-fold change against MDR-K. pneumoniae (FIG. 19B). On the contrary, neither 3-QAC-Hex nor 1-QAC-Hex displayed more than a 2-fold change in MICs against MDR-P. aeruginosa and MDR-K. pneumoniae, respectively (FIG. 19). It was concluded that neither bacteria developed resistance to the 3-QAC-Hex or 1-QAC-Hex polymers after prolonged exposure over 14 passages. Possibly, the membrane targeting ability of both 3-QAC-Hex and 1-QAC-Hex polymers prevented bacteria from gaining a high level of resistance and kept the polymers as active antimicrobial agents after substantial bacterial exposure.

While certain embodiments of the disclosed subject matter have been described using specific terms, such description is for illustrative purposes only, and it is to be understood that changes and variations may be made without departing from the spirit or scope of the subject matter.

What is claimed is:

1. An antimicrobial polymer comprising:
a polymeric moiety;
an aromatic moiety comprising a naphthoic acid derivative bonded to the polymeric moiety;
a pendant group comprising a cation; and
a linking group bonded to the pendant group and aromatic moiety.

2. The antimicrobial polymer of claim 1, wherein the cation comprises an organic cation.

3. The antimicrobial polymer of claim 1, wherein the cation comprises a quaternary ammonium, phosphonium, or sulfonium.

4. The antimicrobial polymer of claim 1 having a molecular weight of 1,000 g/mol to 100,000 g/mol.

5. The antimicrobial polymer of claim 1, wherein the polymeric moiety comprises poly(methyl methacrylate).

6. The antimicrobial polymer of claim 1, wherein the polymeric moiety comprises a polyester, polyamide or polypeptide.

7. The antimicrobial polymer of claim 1, wherein the polymeric moiety comprises a polyalkane.

8. The antimicrobial polymer of claim 1, wherein the antimicrobial polymer comprises a plurality of pendant groups.

9. The antimicrobial polymer of claim 1, wherein the antimicrobial polymer has a selectivity index of between 15 and 1000.

10. The antimicrobial polymer of claim 1, wherein the antimicrobial polymer has a hemolysis of 50% of healthy cells greater than 800 μg/mL.

11. A method for forming an antimicrobial polymer, the method comprising:
reacting naphthoic acid or a derivative thereof with a polymer to form a primary intermediate;
reacting an alcohol group on the primary intermediate with an acyl halide to form a secondary intermediate;
reacting a terminal halide on the acyl halide to form a cationic group on the secondary intermediate.

12. The method of claim 11, wherein a catalyst is present during the step of reacting of the naphthoic acid or a derivative thereof with the polymer.

13. The method of claim 11, wherein the acyl halide comprises a primary chain length of three to eight carbons.

14. The method of claim 11, wherein the cationic group comprises a quaternary ammonium, phosphonium, or sulfonium.

15. The method of claim 11, wherein the naphthoic acid or derivative thereof reacts with an epoxide on the polymer to form the primary intermediate.

16. An antimicrobial medicine comprising the antimicrobial polymer of claim 1.

17. An antimicrobial wound dressing comprising the antimicrobial polymer of claim 1.
